(19) United States
(12) Patent Application Publication
(10) Pub. No.: US 2007/0179454 A1
(43) Pub. Date: Aug. 2, 2007

(54) Safety needle assembly with correct medication connection

(75) Inventors: Lynn A. Ziman, Swanzey, NH (US); Steven Huu Nguyen, North Brunswick, NJ (US)

Correspondence Address: LOUIS WOO;LAW OFFICE OF LOUIS WOO, 717 NORTH FAYETTE STREET, ALEXANDRIA, VA, 22314, US

(73) Assignee: Smiths Medical ASD, Inc., Keene, NH (US)

(21) Appl. No.: 11/342,620

(22) Filed: Jan. 31, 2006

(51) Int. Cl. A61M 5/00

(52) U.S. Cl. 604/249

(57) ABSTRACT

To ensure that there is no misconnection between a needle, such as for example a spinal or epidural needle, and the appropriate medication, the needle hub the needle is formed, by molding or extrusion, to have a given configuration. An adapter is placed between the needle and a conventional fluid store, or medical line. The end of the adapter that connects to the needle assembly is formed to have a configuration that is complementary to that of the hub of the needle assembly, so that the adapter and the needle hub may be readily mated to each other. The other end of the adapter is formed to have another configuration, for example a conventional luer, that is readily connectable to a conventional fluid store, such as a syringe that has a conventional luer. The adapter is formed by inter-fitting two elements to effect an integral locking mechanism to prevent the uncoupling of the fluid store and the adapter, once they are securely coupled. By thus configuring the hub of the needle to have a given configuration that is matable only to the end of an adapter that has a complementary configuration, and further providing an adapter that prevents the removal of the fluid store connected thereto, the potential for mis-connecting the needle to a fluid store that contains a different medicament is greatly reduced, if not totally eliminated.

### FIELD OF THE INVENTION

The present invention relates to a needle assembly, such as for example a spinal or epidural needle assembly, and more particularly to a needle assembly that is designed to mate with an adapter for correct connection to a particular medication store or line.

### BACKGROUND OF THE INVENTION

To prevent mis-connection of a fluid line or a fluid store to a needle, the prior art teaches the use of a two-part connector with complementary configured opposing surfaces. Such two-part connector ensures that one line from one portion of the connector would not be wrongly connected to a different medication line, and is disclosed in U.S. Pat. No. 6,612,624 and its parent U.S. Pat. No. 6,402,207. The disclosure of the '624 patent is incorporated by reference herein.

An adapter that may be used in place of the connector disclosed in the '624 patent is disclosed in U.S. application Ser. No. 10/915,574 filed on Aug. 11, 2004, assigned to same assignee as the instant application. The disclosure of the '574 application is incorporated by reference herein.

As the connector of the '624 patent requires that there be a two-part connection, in order to use a needle, there is the requirement that a fluid line that converts the input of the needle be added. This is an inconvenience, not to mention the consumption of valuable time, in a medical environment where potentially every second counts.

### SUMMARY OF THE PRESENT INVENTION

The instant invention needle assembly includes a needle that has a needle hub specifically configured to have formation(s) thereon that allows it to be connected only to one end of an adapter, which is configured to have a complementary configuration that allows it and the needle hub to readily mate with each other. The other end of the adapter has a conventional receptacle end, which may be in the form of a luer that allows it to be connected to a conventional luer fitted medication store, such as for example a syringe or a medication fluid line. The hub of the needle assembly of the instant invention, as it is configured to have a particular configuration, is not fittable to a conventional luer. Accordingly, the needle assembly could not be mistakenly connected to a fluid store that may contain medicament that, if injected to a patient, may cause harm to the patient.

As the needle may need to be removed from the fluid store, and/or additional medication be provided to the patient, to ensure that the proper medicament is provided to the patient, the adapter that connects the needle to the appropriate medicament store (or fluid line) has a lock mechanism that prevents the removal of the fluid store once the correct fluid store is connected to the adapter. This ensures that no more medication than necessary be injected to the patient, and also that the correct medicament be provided to the patient from the correct mating of the medicament store and the needle.

The lock mechanism for the instant invention is an integral part of the adapter in that the adapter is made up of two components, namely an adapter core and a shroud that fits about the adapter core. The adapter core is fitted to the shroud during manufacturing. The adapter core has a pair of pawls formed at a substantially central portion of its circumferential outer surface or wall. These pawls act against ramped stops formed on the interior circumferential surface or wall of the shroud, when the adapter core and the shroud are rotated relative to each other. Once coupled to the adapter, the fluid store (or fluid line) may no longer be removed from the adapter, as rotation in the direction that ordinarily would have uncoupled the receptacle end of the fluid store from the adapter would cause the receptacle end of the adapter to rotate in unison with the fluid store, thereby preventing the receptacle end of the fluid store and the receptacle end of the adapter from disengaging. As a result, whatever medicament stored in the fluid store that is meant to be used with the needle, which would only mate with the particular adapter, could only be used with the needle, thereby preventing any possible mis-connection of a different medicament container to the needle, which may still be inserted to the patient.

The instant invention therefore relates to an apparatus that comprises a needle assembly that includes a hub having a given configuration at its receptacle end and a needle extending from its closed end, and an adapter having a first end with a first configuration complementary to the given configuration for mating with the hub at its receptacle end. The adapter further has a second end with a second configuration so that a fluid store, or a fluid line, that has a receptacle end with a configuration complementary to the second configuration is adaptable to mate with the second end of the adapter.

The instant invention also relates to a combination in which a needle assembly having a needle hub with a receptacle end of a given configuration and a needle extending from its closed end is combined with an adapter having a first end with a configuration complementary to the given configuration so that the needle hub and the first end of the adapter are readily matable with each other. The adapter has a second end with a second configuration that allows the adapter to be coupled to the receptacle end of a fluid store, or a fluid line. The adapter further acts as a locking mechanism to prevent the fluid store (or the fluid line), once coupled to the adapter, from rotating in a direction relative to the adapter that allows the fluid store from being uncoupled from the adapter.

The instant invention further relates to a method of coupling a needle assembly including a hub having a receptacle end and a needle extending from its closed end to an appropriate medicament fluid store or fluid line. The method includes the steps of: a) effecting a given configuration at the receptacle end of the hub; b) providing an adapter with a first end having a first configuration complementary to the given configuration for mating with the hub at its receptacle end; c) effecting a second configuration to a second send of the adapter; and d) providing a fluid store or a fluid line having a receptacle end with a configuration complementary to the second configuration for mating with the second end of the adapter.

### BRIEF DESCRIPTION OF THE FIGURES

The present invention will become apparent and the invention itself will be best understood with reference to the following description of the present invention taken in conjunction with the accompanying drawings, wherein:

FIG. 1 illustrates a needle of the instant invention with an adapter and a fluid store;

FIG. 2 is a view illustrating the disassembled components of the adapter, as it relates to the needle;

FIGS. 3a and 3b are side views of the needle assembly of the instant invention;

FIGS. 4a-4f are different views of the adapter core component of the adapter;

FIGS. 5a-5g are different views of the shroud component of the adapter; and

FIG. 6 shows a filter interposed between the adapter and the needle assembly.

### DETAILED DESCRIPTION OF THE INVENTION

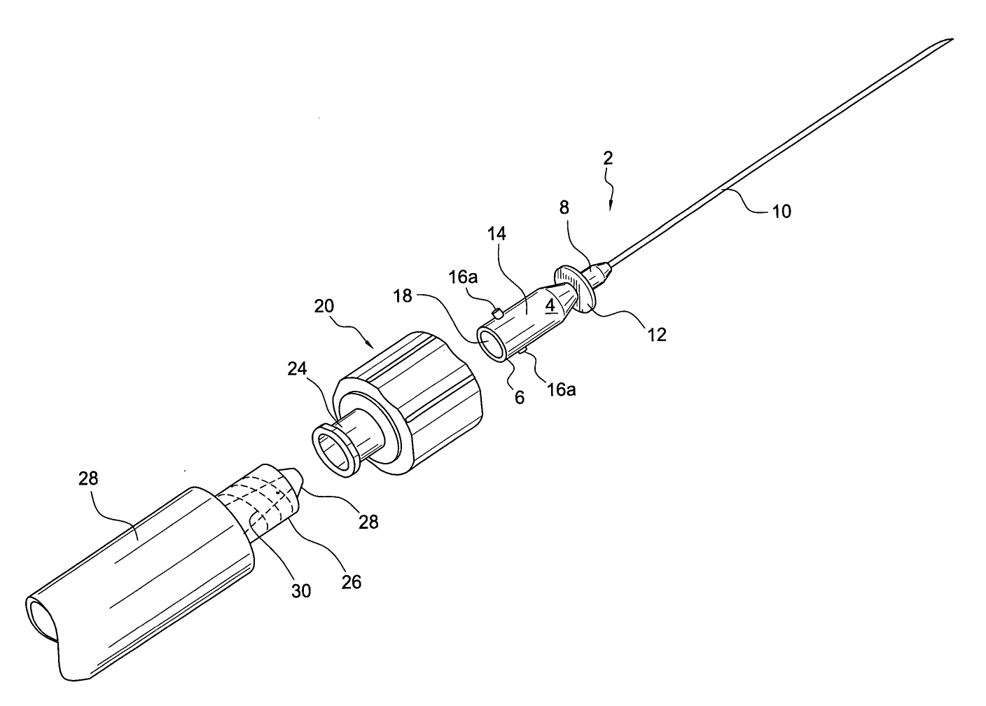

The instant invention, as shown in FIG. 1, includes a needle assembly 2 that has a needle hub 4. Needle hub 4 has a receptacle end 6 and a closed end 8 from which a needle 10 extends. Needle 10 may be a conventional needle, but for the instant embodiment is an epidural or a spinal needle for insertion to a patient. As shown, the closed end 8 is separated from the rest of needle hub 4 by a flange 12.

Receptacle end of needle hub 4 comprises an elongate cylindrical portion whereat a particular formation, or formations, are effected during the manufacturing process for providing a particular or given configuration that is unique to the needle assembly. For the embodiment shown, there are two protrusions 16a and 16b formed at opposite sides of the receptacle end 6. These protrusions, along with the cross sectional dimension of receptacle end 6, provide the given configuration for needle hub 4 of the needle assembly 2. The thickness of the circumferential wall of the elongate cylindrical portion 14 is dimensioned to provide a further attribute of the configuration of needle hub 4, i.e., configuring the cross section of opening 18 at receptacle end 6 and the through passage into needle hub 4. Opening 18 is of a sufficient dimension to accept the receptacle end of an adapter 20 to which needle hub 4 of the needle assembly 2 mates with. Protrusions 16a and 16b are formed on the receptacle end 6 of needle hub 4 a particular distance from opening 18 as part of the formation for effecting the proper mating with adapter 20, more specifically with the receptacle end thereof and the shroud component of adapter 20, to be discussed later.

As further shown in FIG. 1, adapter 20 has another receptacle end 24, which may be configured as a conventional luer end for mating with the conventional luer end 26 of a fluid store, such as for example a syringe 28. In place of a fluid store, a fluid line having fitted to its end a luer such as 26 is also contemplated. Although the receptacle end 26 of syringe 28 is able to mate with receptacle end 24 of a adapter 20, it could not mate with the receptacle end 6 of needle assembly 2. Receptacle end 26 of syringe 28 has a male receptacle end 28, and is internally threaded, as represented by the dotted threaded line 30.

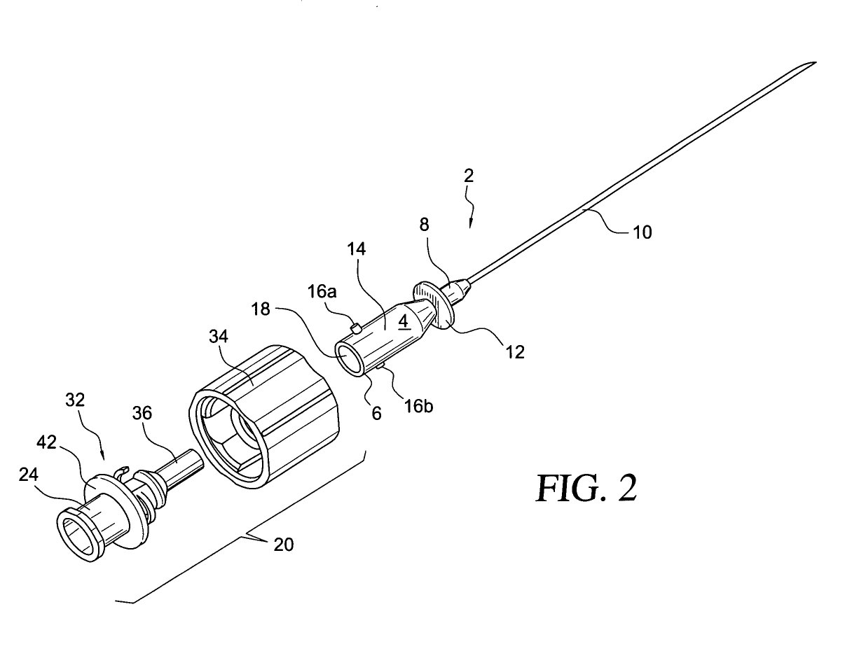

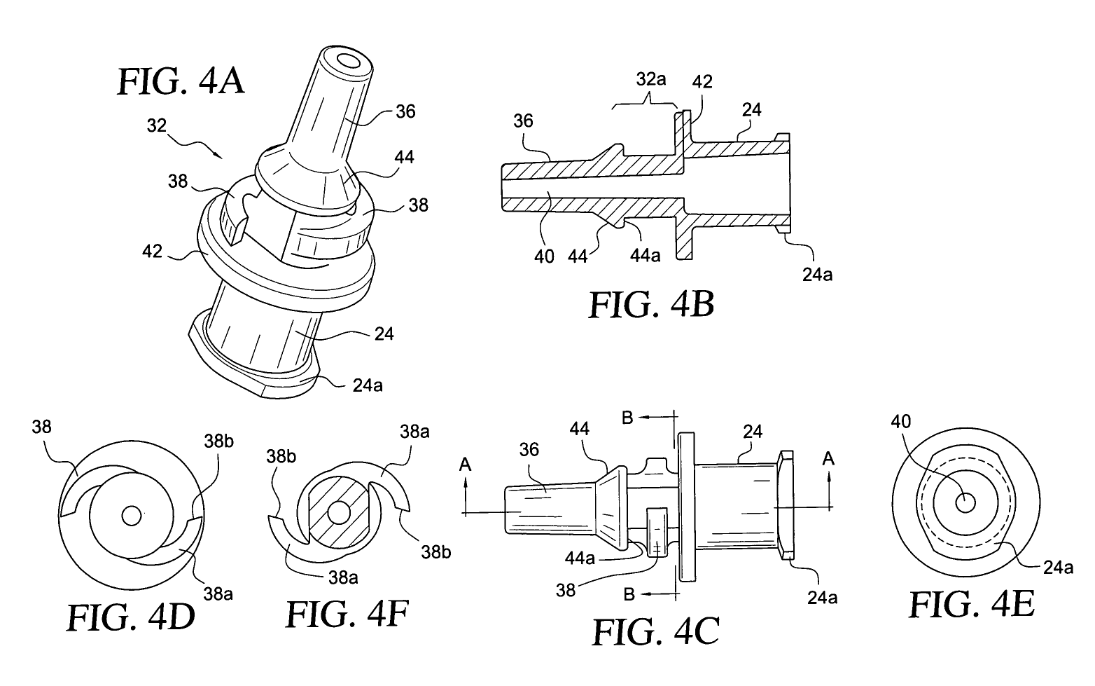

FIG. 2 shows the different components of adapter 20 in relation to needle assembly 2. As shown, adapter 20 comprises an adapter core 32 and a shroud 34 that is adapted to fit about core 32. As mentioned previously, adapter core 32 has a receptacle end 24, which may also be referred to as its second or distal end. In addition, core 32 has a first receptacle end 36, or a first or proximal end, for mating with receptacle end 6 of needle hub 4. The first end 36 and the second end 24 of adapter core 32 are connected, as adapter core 32 is a single molded piece, by a through passage 40 (FIG. 4b) that extends from the opening at first end 36 to the opening at second end 24. Shroud 34 and adapter core 32 are separate pieces that may be molded from conventional medical plastics.

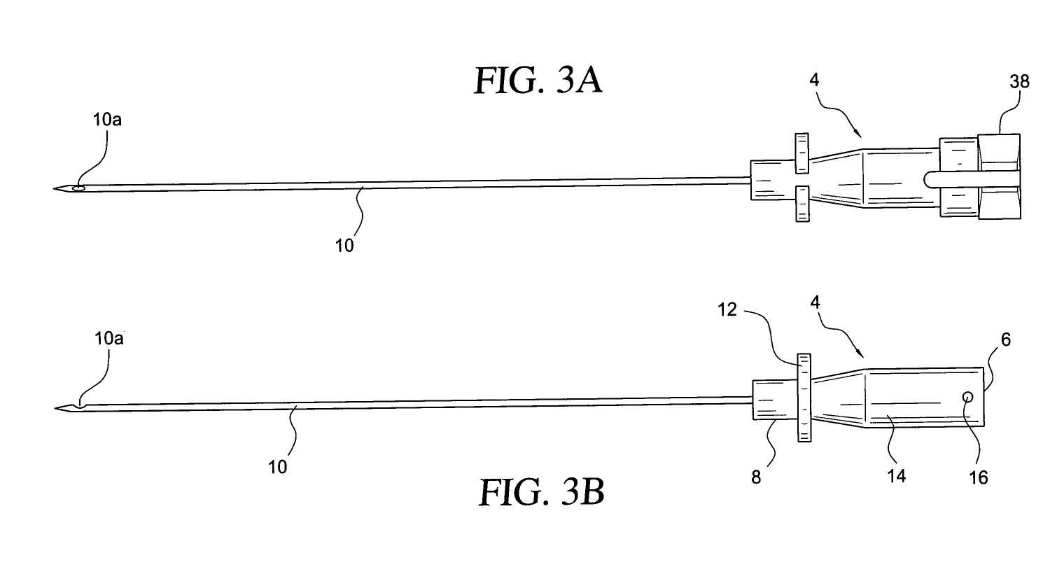

Needle assembly is shown in side views 3a and 3b. FIG. 3a shows the receptacle end 6 of needle hub 4 of the needle assembly 2 being covered by a cap 38 that has extending therefrom a thin cannula (not shown) inserted within needle 10 to prevent coring, or the blocking of the needle opening 10a, when needle 10 is inserted to the patient. It is only after needle 10 has been properly inserted into the patient would the user remove cap 38, and therefore the cannula inserted in needle 10. At which time, fluid may pass between needle 10 and the patient through opening 10a at the tip of needle 10.

Adapter core 32 of adapter 20 of the instant invention, as best shown in FIGS. 4a-4f, has an elongate conical first end 36 and a cylindrical second end 24, which has at its end flanges 24a for forming a conventional luer connection. As best shown in FIGS. 4a, 4d and 4f, at the proximate mid-section of adapter core 32 there are two integral pawls 38 formed at the outer circumferential surface or wall of core 32. These pawls each have a semi-ramped portion 38a and an end stop 38b, as best shown in FIG. 4f. As best shown in FIG. 4b, a through passage 40 extends from the opening at first end 36 to the opening at second end 24. A flange 42 that separates the first end from the second end provides a back stop, when adapter core 36 is fitted into shroud 34 (or conversely shroud 34 being fitted about adapter core 32), as shown in FIG. 1. A tapered circumferential portion 44 enables adapter core 32 to be inserted to shroud 34, and be fixedly but rotatably retained within shroud 34.

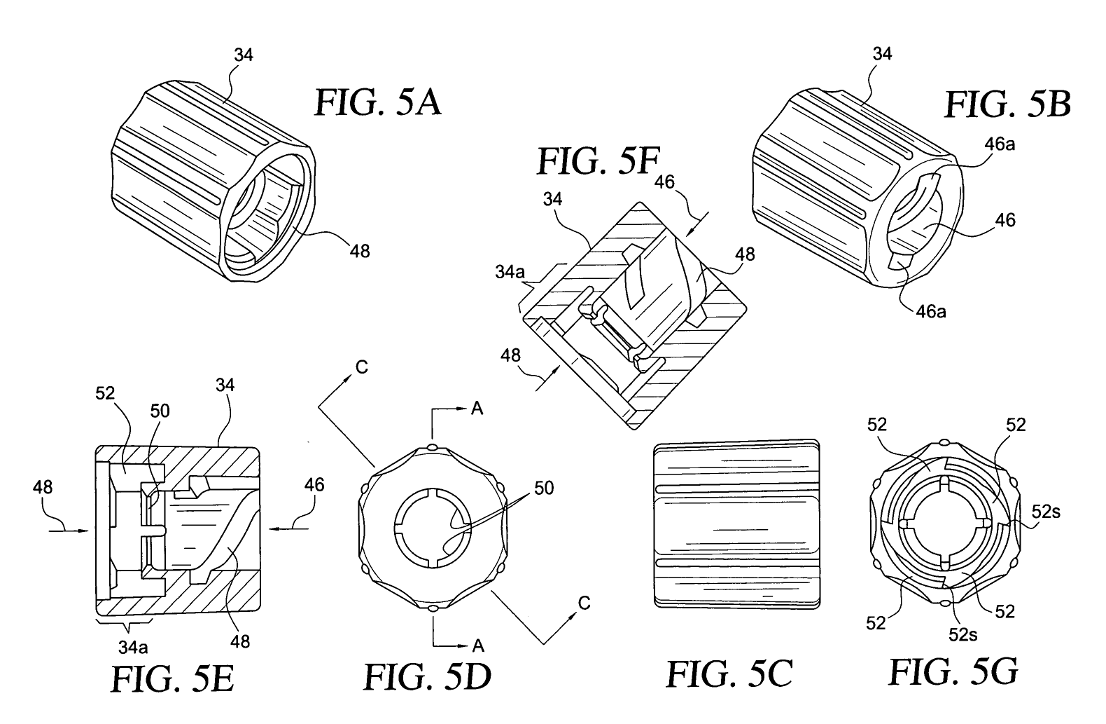

Shroud 34 is shown in FIGS. 5a-5g to have a first opening 46 and a second opening 48. Opening 46 is fabricated to have a configuration that is complementary to the configuration of the formation at receptacle end 6 of needle assembly 2, as discussed above. In particular, as shown in FIG. 5b, opening 46 of shroud 34 has a formation in the shape of a circle, but with two side inlets or channels 46a that allow the corresponding protrusions 16 of needle hub 4 to pass through. As the shroud is formed with an internal thread 48, receptacle end 6 of needle hub 4, once inserted to opening 46, may be secured to shroud 34 by the relative rotation of hub 4 and shroud 34, so that protrusions 16 of needle hub may travel along the internal thread of shroud 34, and receptacle end 6 via its opening 18 be more fully mated with the receptacle end 36 of adapter core 32. Once fully threaded, needle hub 4 is securely mated to adapter 20.

Adapter core 32 is pressedly fitted to shroud 32 in the direction as shown by directional arrow 48 in FIGS. 5e and 5f. The portion of adapter core 32, designated 32a in FIG. 4b, is fitted and held by shroud 34 at portion 34a, as shown in FIGS. 5e and 5f. A number of extensions 50 circumferentially spaced at the interior wall of shroud 34, due to their inherent plastics elasticity, would allow portion 44 of adapter core 32 to pass through, as the latter's front is tapered, but would prevent the same from coming back out of shroud 34, as the circular flat backside 44a of portion 44 is biasedly held by extensions 50. As a result, once adapter core 32 is fitted into shroud 34, it cannot be removed therefrom.

Also provided in shroud 34 are a number of ramped stops 52, as best shown in FIG. 5g, that prevent adapter core 32 from rotating in a direction where the stop surfaces 52s would bias against pawl stops 38b. In practice, if a user were to hold adapter 34 by its receptacle end 24 while attempting to rotate shroud 34, she could only rotate shroud 34 in a counterclockwise direction. Any attempt to rotate shroud 34 relative to adapter core 32 in the clockwise direction would also cause adapter 32 to rotate in unison with shroud 34.

In operation, by rotating luer end 24 of adapter 20 relative to the luer end of a fluid store, such as for example syringe 28 shown in FIG. 1, in a clockwise direction, adapter 20 can readily be coupled to the fluid store. However, any attempt to remove adapter 20 from syringe 28, by for example rotating adapter 2 (more specifically shroud 34) counterclockwise relative to syringe 28 would fail, as adapter core 32 would move (rotation or non-rotation) in unison with syringe 28, while the movement of shroud 34 (non-rotation or rotation) would be independent of the unified movement of adapter core 32 and syringe 28. As a result, once syringe 28 is coupled to adapter 20, it remains coupled thereto, thereby eliminating the real possibility that a different syringe may be mis-connected to needle assembly 2, which hub has a configuration that could only mate with the complementary configuration at receptacle end 36 of adapter 20.

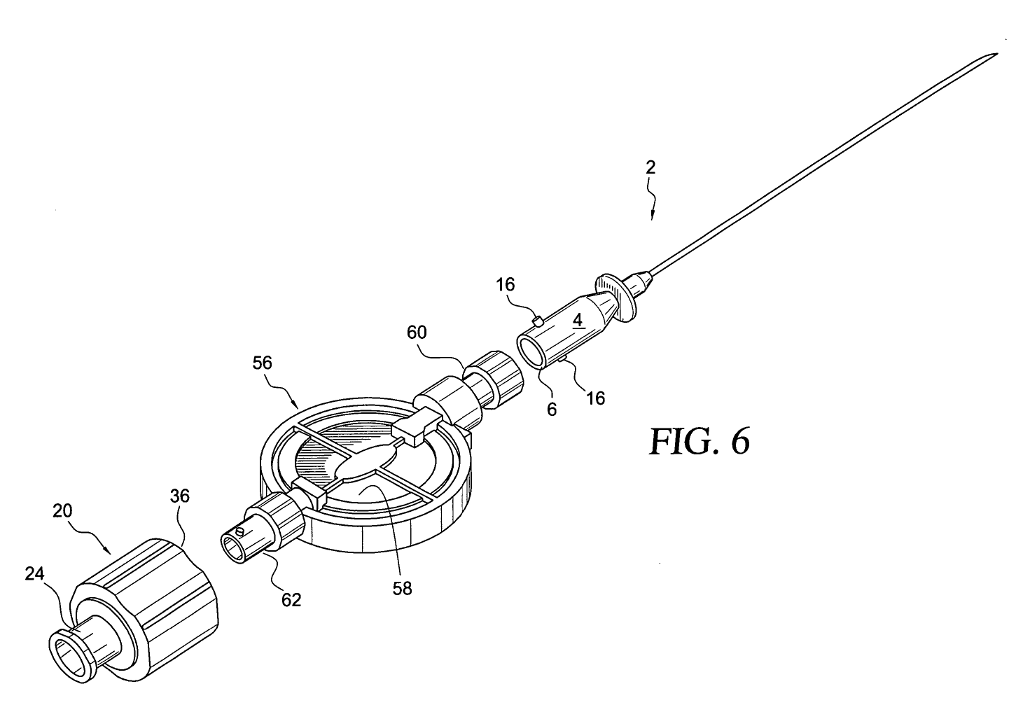

Another aspect of the instant invention is shown in FIG. 6. There a filter device 56 is shown to be interposedly connectable to adapter 20 and needle assembly 2. Filter device 56, in addition to having a filter element 58, has a first end 60 that has a configuration that is complementary to the configuration of the receptacle end 6 of needle hub 4 of the needle assembly 2. In other words, the configuration of the receptacle end 60 for filter device 56 is the same as the configuration of the receptacle end 36 of adapter 20. Further, filter device 56 has a second end 62 for mating with adapter 20 that has a configuration that is the same as the configuration of receptacle end 6 of needle hub 4. Once fully connected to both needle assembly 2 and adapter 20, filter device 56 filters the fluid passing between the needle assembly 2 and adapter 20, and of course the fluid store that is connected to end 24 of adapter 20. The filter element 58 of filter device 56 may be any conventional medical filter that is adaptable to filter out undesirable particles or elements that may be in the fluid.

1. Apparatus comprising:
a needle assembly including a hub having a given configuration at its receptacle end and a needle extending from its closed end;
an adapter having a first end with a first configuration complementary to said given configuration for mating with said hub at its receptacle end, said adapter having a second end with a second configuration; and
a fluid store or a fluid line having a receptacle end with a configuration complementary to said second configuration for mating with the second end of said adapter.

2. Apparatus of claim 1, wherein said adapter comprises a locking mechanism to prevent the removal of said fluid store or fluid line from said adapter once said fluid store or fluid line and said adapter are mated to each other.

3. Apparatus of claim 1, wherein the receptacle end of said hub has an integral formation for forming said given configuration so that said hub is adapted to mate only with a counterpart receptacle end that has a configuration complementary to said given configuration.

4. Apparatus of claim 3, wherein said integral formation of the receptacle end of said hub comprise two protrusions extending from the receptacle end of said hub.

5. Apparatus of claim 2, wherein said adapter comprises a shroud fitted about an adapter core, the first and second ends of said adapter formed at opposite ends of said adapter core, at least one pawl formed on the circumferential outer surface of said adapter core for coacting against ramped stops formed circumferentially along the interior surface of said shroud so that once said fluid store or fluid line is mated to the second end of said adapter, said fluid store or fluid line and said adapter cannot be uncoupled from each other.

6. Apparatus of claim 1, wherein said needle assembly comprises an epidural needle.

7. Apparatus of claim 1, wherein said adapter comprises an adapter core having a through channel and a shroud fitted about said adapter core, the first end of said adapter core being a tapered inner receptacle end that forms the first end of said adapter, said shroud having opposed inlets to an internal thread for threadedly mating with the receptacle end of said hub as the receptacle end of said hub is fitted to the tapered inner receptacle end of said adapter core.

8. Apparatus of claim 1, wherein said first configuration of the receptacle end of said adapter is not connectable to a needle assembly having a conventional luer.

9. Apparatus of claim 1, further comprising a filter having a first end with a configuration complementary to said given configuration of said hub and a second end having the same said given configuration as said hub, said filter adapted to be connectably interposed between said needle assembly and said adapter by having its first end coupled to the receptacle end of said hub and its second end coupled to the first end of said adapter.

10. In combination, a needle assembly having a needle hub with a receptacle end of a given configuration and a needle extending from its closed end; an adapter having a first end with a configuration complementary to said given configuration so that said needle hub and the first end of said adapter are readily matable with each other, said adapter having a second end with a second configuration that enables said adapter to be coupled to the receptacle end of a fluid store or a fluid line, said adapter including a locking mechanism that prevents said fluid store or fluid line, once coupled to said adapter, to rotate in a direction relative to said adapter that allows said fluid store or fluid line from being removed from said adapter.

11. Combination of claim 10, wherein said the receptacle end of said hub is cylindrically shaped, and said given configuration of said cylindrical receptacle end is formed by an integral formation; and
wherein the first end of said adapter has a counterpart complement to said integral formation.

12. Combination of claim 11, wherein said integral formation comprises two protrusions at opposite sides of said cylindrical receptacle end of said hub.

13. Combination of claim 10, wherein said adapter comprises an adapter core and a shroud fitted about said adapter core, the first and second ends of said adapter formed at opposite ends of said adapter core, at least one pawl formed on the circumferential outer surface of said adapter core for coacting against ramped stops formed circumferentially along the interior surface of said shroud so that once said fluid store or fluid line is mated to the second end of said adapter, said fluid store or fluid line can no longer be uncoupled from said adapter.

14. Combination of claim 10, wherein said needle comprises an epidural needle.

15. Combination of claim 10, further comprising a filter having a first end with a configuration complementary to said given configuration of said hub and a second end having the same said given configuration as said hub, said filter adapted to be connectably interposed between said needle assembly and said adapter by having its first end coupled to the receptacle end of said hub and its second end coupled to the first end of said adapter to provide a filtered fluid communication path between said needle and said adapter.

16. A method of coupling a needle assembly including a hub having a receptacle end and a needle extending from its closed end to an appropriate medicament fluid store or fluid line, comprising the steps of:
a) effecting a given configuration at the receptacle end of said hub;
b) providing an adapter having a first end with a first configuration complementary to said given configuration for mating with said hub at its receptacle end;
c) effecting a second configuration to a second end of said adapter; and
d) providing a fluid store or a fluid line having a receptacle end with a configuration complementary to said second configuration for mating with the second end of said adapter.

17. Method of claim 16, wherein said step a further comprises the step of:
providing an integral formation at the receptacle end of said hub to form said given configuration so that said hub is adapted to mate only with a counterpart receptacle end that has a configuration complementary to said given configuration.

18. Method of claim 16, further comprising the step of:
providing a locking mechanism at said adapter to prevent the removal of said fluid store or fluid line from said adapter once said fluid store or fluid line and said adapter are mated to each other.

19. Method of claim 16, wherein said step b further comprises the steps of:
providing an adapter core and a shroud fittable about said adapter core as parts of said adapter;
forming the first and second ends of said adapter at opposite ends of said adapter core;
forming at least one pawl on the circumferential outer surface of said adapter core and ramped stops circumferentially along the interior surface of said shroud;
rotatably fitting said shroud about said adapter core to form said adapter;
wherein once said fluid store or fluid line is mated to the second end of said adapter, the pawl of said adapter core would coact with the ramped stops of said shroud to enable said fluid store or fluid line to be rotatable only in a direction that does not allow said adapter and said fluid store or fluid line to be uncoupled from each other.

20. Method of claim 16, further comprising the step of:
connectably interposing a filter having a first end with a configuration complementary to said given configuration of said hub and a second end having the same said given configuration as said hub between said needle assembly and said adapter by coupling the first end of said filter to the receptacle end of said hub and the second end of said filter to the first end of said adapter to thereby effect a filtered fluid communication path between said needle and said adapter.
